(19) United States
(12) Patent Application Publication
(10) Pub. No.: US 2026/0076744 A1
(43) Pub. Date: Mar. 19, 2026

(54) COMPUTER-BASED METHODS AND SYSTEMS FOR DIGITALLY ADJUSTING JOINT LAXITY IN A PERSONALIZED SURGICAL PLAN OF A JOINT ARTHROPLASTY PROCEDURE

(71) Applicant: EXACTECH, INC., Gainesville, FL (US)

(72) Inventors: Laurent Angibaud, Gainesville, FL (US); Prudhvi Chinimilli, Milpitas, CA (US)

(21) Appl. No.: 19/329,045

(22) Filed: Sep. 15, 2025

Related U.S. Application Data
(60) Provisional application No. 63/695,120, filed on Sep. 16, 2024.

(51) Int. Cl. A61B 34/10; A61B 5/11; A61B 17/02; A61B 34/00; A61B 34/30; A61B 90/00

(52) U.S. Cl. CPC A61B 34/10; A61B 5/1121; A61B 17/025; A61B 34/25; A61B 34/30; A61B 90/06; A61B 90/36; A61B 2017/0268; A61B 2034/104; A61B 2034/105; A61B 2034/107; A61B 2034/252; A61B 2034/256; A61B 2090/066; A61B 2090/374; A61B 2090/3762

(57) ABSTRACT

A method is provided for facilitating the implantation of an implant during a surgical procedure. The method includes obtaining bone registration data for a first bone member and a second bone member of a patient's joint, and acquiring patient-specific movement-related data during the procedure after the joint is subjected to at least one movement with a first predefined distraction force applied throughout a continuous range of motions. A distraction force model is used to conduct a joint gap simulation based on predefined ligament stiffness data, the movement-related data at the first distraction force, and at least one simulation distraction force value, resulting in joint gap simulation data. A surgical plan model utilizes the joint gap simulation data and bone registration data to generate a patient-specific intra-operative surgical plan comprising at least one surgical cut parameter. The method further includes cutting the first and/or second bone member based on the surgical plan to facilitate implant implantation.

### FIELD OF TECHNOLOGY

The present disclosure generally relates to orthopedic surgery, and more particular to an computer-based methods and systems for digitally adjusting joint laxity in a personalized surgical plan of a joint arthroplasty procedure.

### BACKGROUND OF TECHNOLOGY

Total knee arthroplasty (TKA) is a well-established procedure for end-stage osteoarthritis of the knee joint, which may result in relief of pain and improved physical function. However, problems still need to be solved for achieving desired long-term functional results and patient satisfaction. Knee instability after primary TKA is an essential factor in early TKA failure. Studies have shown that knee instability may be due to an inadequate correction of soft tissue imbalance in the sagittal, coronal, and axial planes. As a result, soft tissue balancing is needed for improving the outcomes of TKA.

Historically, the TKA procedure was based on maintaining a hip-knee-ankle (HKA) angle of 180° by positioning both the tibial and femoral components perpendicular to the mechanical axis of each bone. In this mechanical alignment (MA) method, coronal alignment is prioritized, and the same bone cuts may made in every knee, regardless of unique patient characteristics or deformity. After the completion of the bone cuts, a soft tissue release may be performed as needed to balance the soft tissue envelope and to create symmetric flexion and extension gaps. However, the ligament release stage is highly subjective where the experience of the surgeon is crucial. Thus, a lack of optimal soft balance may cause suboptimal results in terms of TKA patient satisfaction. Thus, there is a need in the art for surgical workflows that facilitate soft tissue balancing.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 schematically illustrates an operating room using an improved computer-based surgery assistance device for digitally adjusting joint laxity in a personalized surgical plan of a joint arthroplasty procedure in accordance with one or more embodiments of the present disclosure;

FIG. 2 is a block diagram of a controller of an improved computer-based platform for digitally adjusting joint laxity in a personalized surgical plan of a joint arthroplasty procedure in accordance with one or more embodiments of the present disclosure;

FIG. 3 shows an exemplary embodiment of a distractor in accordance with one or more embodiments of the present disclosure;

FIGS. 4A and 4B illustrate a distractor inserted between a femur and a tibia after a femoral cut in accordance with one or more embodiments of the present disclosure;

FIG. 5 is a graph of laxity curves for different force environments in accordance with one or more embodiments of the present disclosure;

FIG. 6 is a graph showing a first set of joint gaps based on physical acquisitions using a first distraction force and a personalized second set of joint gaps based on a digital application of a second distraction force in accordance with one or more embodiments of the present disclosure;

FIG. 7 is a graph that defines a stiffness as a slope of the elongation curve in accordance with one or more embodiments of the present disclosure;

FIGS. 8A-8D are graphs illustrating knee joint medial and lateral stiffness factors calculated from cadaver-based test data in accordance with one or more embodiments of the present disclosure;

FIG. 9 are plots showing knee torque versus knee angle and knee torque versus knee angular velocity for a participant under different walking conditions in accordance with one or more embodiments of the present disclosure;

FIGS. 10A and 10B illustrate exemplary embodiments of a knee dynamometer and/or rotometer apparatus in accordance with one or more embodiments of the present disclosure;

FIG. 11 illustrates the processes for musculoskeletal multibody simulations of a lower extremity with a total knee replacement using a motion capture system in accordance with one or more embodiments of the present disclosure; and

FIG. 12 is a flowchart of an exemplary method for digitally adjusting joint laxity in a personalized surgical plan of a joint arthroplasty procedure in accordance with one or more embodiments of the present disclosure.

### SUMMARY

In at least some embodiments, the present disclosure provides a technically improved computer-implemented method for patient-specific implantation of an implant in a joint that may include the following steps: initiating a surgical procedure; obtaining bone registration data for first and second bone members of the joint; acquiring patient-specific movement-related data during at least one movement under a predefined distraction force applied continuously across the joint throughout a range of motion; conducting a joint gap simulation with a distraction force model based on predefined ligament stiffness data, the patient-specific movement data, and one or more simulated distraction force values; obtaining joint gap simulation results; using a surgical plan model to generate a patient-specific intra-operative surgical plan that includes at least one surgical cut parameter based on the simulation data and the bone registration data; and performing bone cuts on one or both bone members according to the surgical cut parameter to facilitate implant implantation. The method may further include measuring joint gaps at multiple flexion angles throughout the range of motion; applying the predefined distraction force with a distractor device that maintains a substantially constant axial force; determining ligament stiffness by linear regression analysis of load versus joint gap data; and selecting predefined ligament stiffness values from a database indexed by patient demographic information.

In at least some embodiments, the present disclosure provides a technically improved computer-based system that may include at least the following components: at least one memory for storing instructions; and at least one processor in communication with the at least one memory, the at least one processor may be configured to execute the instructions to: acquire patient-specific movement-related data during at least one movement under a predefined distraction force applied continuously across the joint; simulate joint gaps using a distraction force model based on predefined ligament stiffness data, the patient-specific movement data, and simulated distraction force values; generate a patient-specific intra-operative surgical plan via a surgical plan model that specifies at least one surgical cut parameter; and control a surgical apparatus to perform bone cuts on one or both bone members according to the surgical cut parameter. The system may further include a distractor device to apply and maintain a substantially constant axial force; a motion capture system to obtain movement-related measurements such as joint torque and angular velocity after subjecting the joint to multiple spatial positions; a database of predefined ligament stiffness values indexed by patient demographic information; and a graphical user interface to display the surgical plan and real-time graphical representations of simulated joint gaps to the surgeon. The system may also be configured for real-time generation and updating of the patient-specific intra-operative surgical plan, assignment of distinct stiffness values to soft-tissue portions including medial and lateral collateral ligaments, and generation of multiple surgical plans for varied simulated distraction force scenarios for direct comparison.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “In at least some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In at least some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

Difficulties in performing efficient soft tissue balancing during total knee joint arthroplasty (TKA) procedures may be a major factor that causes suboptimal results in terms of long term TKA patient satisfaction. To solve this technical problem, advanced algorithmic-based surgical assistance techniques may utilize the acquisition of the joint laxity (or joint gap) data under a known distraction force as an input for the set-up of the surgical planning for defining bone cut parameter(s) based on alignment, size (e.g., fit with the bone), as well as laxity considerations.

Instead of only acquiring joint laxity data using a known quasi-constant distraction force such as 90 N that may be independently applied to each compartment of the knee joint, or a distraction force of 180 N (i.e., 90 N per compartment) for the final set-up of the surgical planning for defining bone cut parameter(s), at least some embodiments of the present disclosure herein describe the use of digital simulated supplemental distraction force options as an input for the acquisition of simulated joint laxity, or joint gaps, as an output for the set-up of the surgical planning.

The underlying reasons for using different force options in surgical planning may include:
Surgeon-specific rationale: Depending on their training, and/or experiences, and/or geographic area, surgeons may prefer lower or higher joint loading scenarios than the one offered by a distractor device configured to apply a distraction force.
Patient-specific rationale: Depending on the stature, and/or the activity expectation, and/or the pathology (e.g., hypermobility/hyperlaxity vs. stiffness), the patient may benefit from a more personalized distraction force.
Flexion angle and/or compartment rationale: depending on the joint flexion angle, the load patterns tend to fluctuate between the medial and the lateral compartments.

Thus, systems and methods for providing multiple distraction force options may require a set of multiple distractor/tensioner devices, where each individual device would provide a specific distraction force [Option 1] or a distractor/tensioner device able to implement suitable adjustments leading to the setup of different distraction force options [Option 2]. Implementation of Option 1 may be costly (e.g., cost of numerous devices vs. a single device) and/or time-consuming (e.g., time to perform micro adjustment vs. ready-to-use device), and inevitably limited in terms of force options. Implementation of option 2 may be costly and complex since a distractor device having tunable distraction force capabilities may be bulky and heavy with a need for wire management.

At least some embodiments of the present disclosure as described herein enable the full personalization of the force environment as an input to acquire the joint laxities (e.g., joint gaps) to be leveraged for the definition of the bone cut parameters as part of the intra-operative surgical planning of an enabling technology.

At least some of the embodiments described herein may be applied to any joint surrounded by soft-tissue (e.g., knee, hip, ankle, shoulder, . . . ) and any type of arthroplasty (i.e., resurfacing, partial, or total) of the joint.

At least some of the embodiments described herein may further encompass the possibility of physically applying a first known distraction force as an input to a joint of a patient to acquire a first definition of the physical joint gaps as an output, then to combine a personalized second distraction force environment with an assumed stiffness (e.g., defined as the slope of the load-elongation curve or other method) of the soft-tissue surrounding the considered joint to simulate a second definition of the joint gaps to be used for the definition of the bone cut parameters of the considered joint during the intra-operative surgical planning process.

In at least some embodiments, the digital enhancement of the laxity curves may allow for the implementation of an infinite combination of force options through the sole usage of a single instrument intended to apply a first known force as a reference.

In at least some embodiments, the technical advantage of the technical solutions described herein may relate to the possibility of simulating different surgical plans based on different sets of joint laxities obtained under different simulated distraction force environments. Thus, this may provide a surgeon user to the possibility to compare these different surgical plans before the execution of the bone cuts.

In at least some embodiments, the technical advantage of the technical solutions described herein may allow for the recording of the personalized force as part of the technical log associated with the enabling technology, information needed for the understanding of the impact of the distraction force on clinical outcomes.

In at least some embodiments, the second distraction force may be fully personalized to the patient and/or surgeon's preference by allowing the simulation of unlimited distraction force scenarios after the usage of a single distraction force device.

At least some of the embodiments in the present disclosure herein describe an improved computer-based platform for digitally adjusting joint laxity in a personalized surgical plan of a joint arthroplasty procedure. The computer-assisted surgery (CAS) platform reduces intra-operative technical errors during joint arthroplasty procedures and accounts for the proper management of the soft-tissue surrounding the joint as an important factor to improve patient satisfaction as well as clinical outcomes. Ligament balancing techniques, measured and/or algorithmic-based, in different force regimes may be incorporated into a patient-specific surgical plan for improving total knee arthroplasty (TKA) outcomes, for example. Moreover, CAS technologies may be used to provide guidance to the surgeon both before and during the total arthroplasty procedure.

U.S. patent application Ser. No. 18/054,653 filed Nov. 11, 2022, and U.S. patent application Ser. No. 17/349,370 filed Jun. 16, 2021, are incorporated herein by reference in their entirety.

In at least some embodiments, the CAS technologies may be image-based and may incorporate pre-operative computed tomography (CT) scans and/or pre-operative magnetic resonance imaging (MRI) scans of the joint, for use in the determination of a patient-specific surgical plan. Then, a CAS surgical plan software application as described herein may provide pre-operative and/or intra-operative planning. The surgeon may establish a surgical plan not only based on selecting the proper size and type of implant, but also theoretically vary the laxity curves in different force regimes to predict different post-operative outcomes. It should be understood to one skilled in the art that the CAS technologies shown herein may be applied to any joint arthroplasty procedure for any joint in a living body and not limited to a total knee arthroplasty procedure as per the exemplary embodiments shown herein.

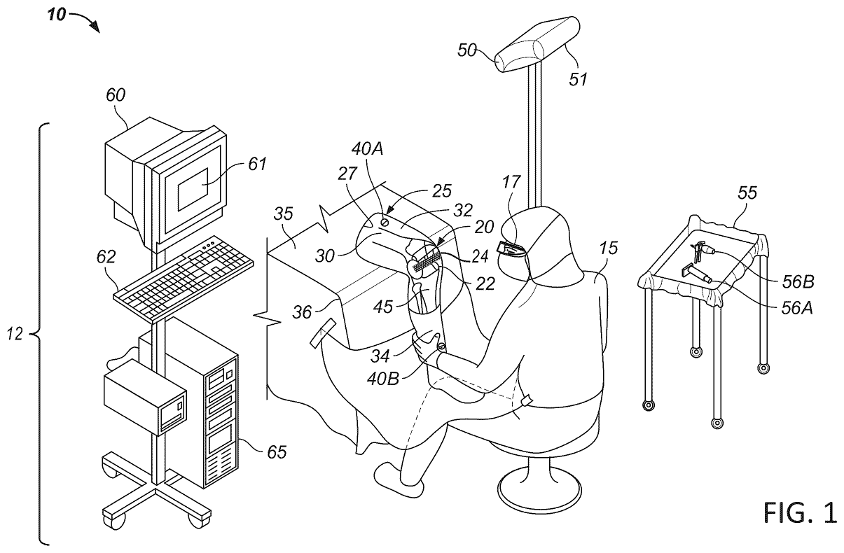

FIG. 1 schematically illustrates an operating room 10 using an improved computer-based surgery assistance device 12 for digitally adjusting joint laxity in a personalized surgical plan of a joint arthroplasty procedure in accordance with one or more embodiments of the present disclosure. The embodiments shown in FIG. 1 refer to a total knee arthroplasty procedure. FIG. 1 shows a surgeon 15 operating on a leg 25 of a patient positioned on an operating table 35. The leg 25 of the patient may be placed through a surgical drape opening 27 for access to the leg 25 by the surgeon 15. In this exemplary embodiment, the surgeon 15 may perform a total knee arthroplasty procedure on the patient via an incision 22 made by the surgeon 15 to expose a knee joint 20 of the patient. A distractor 24 may be placed by the surgeon 15 into the knee joint 20 for soft tissue characterization under an application of at least one distraction force. The leg 25 as shown in FIG. 1 may include an upper portion 32 (e.g., a first member-thigh) with a femur 30 (e.g., first bone member), a lower portion 34 (e.g., a second member-calf) with a tibia 45 (e.g., second bone member), and the knee joint 20.

In at least some embodiments, at least one first tracking device 40A may be coupled to the upper portion 32 of the leg 25 (e.g., a first bone member) and at least one second tracking device 40B may be coupled to the lower portion 34 of the leg 25 (e.g., a second bone member). In other embodiments, the at least one first tracker 40A and the at least one second tracker 40B may be rigidly mounted to the bone members (e.g., respectively to the femur 30 and to the tibia 45 for the embodiments of FIG. 1).

In at least some embodiments, the operating room 10 may include at least one imaging camera 50 shown schematically in FIG. 1 mounted on an image camera assembly 51. Note that any suitable number of cameras of any suitable type may be mounted on the image camera assembly 51 that may be used to track 3D objects. The at least one imaging camera 50 may be used to acquire a position and/or orientation of the bone members in a three-dimensional (3D) environment.

In at least some embodiments, the operating room 10 may include at least one surgical tool 56A and/or at least one surgical probe 56B placed on a cart 55 easily accessible by the surgeon 15 during the joint arthroplasty procedure.

In at least some embodiments, the surgery assistance device 12 in the operating room 10 may include a controller 65, a keyboard 62 and/or a display 60 displaying a graphic user interface (GUI) 61 with graphic user interface elements to allow the surgeon 15 to interact with.

In at least some embodiments, the display 60 may be a screen/monitor directly accessible to the surgeon 15 and/or by a wearable display 17 (e.g., heads up display, smart glasses) directly worn by the surgeon 15 during the surgical procedure to provide a computer-controlled augmented reality view for the surgeon 15. The controller 65 may be communicatively coupled to any of the surgical tools used by the surgeon 15 to perform the total joint arthroplasty.

In at least some embodiments, the controller 65 may display on the GUI 61 of the display 60, a patient-specific surgical plan to assist the surgeon 15 to perform the placement of the joint implant into the joint of the patient undergoing the total joint arthroplasty. The keyboard 62 may be used by the surgeon 15 or any other medical personnel assisting the surgeon 15 to input patient-specific data into the controller 65 via the keyboard 62 either before and/or during the joint arthroplasty procedure such that the algorithms executed by the controller 65 may generate and/or update the surgical plan in real time so as to assist the surgeon 15 before and/or during the joint arthroplasty procedure. The GUI 61 may further allow the surgeon 15 to vary the distraction force regimes to view the impact of changes in the soft tissue balancing of the joint for proper pre-operative and/or intra-operative surgical planning.

In at least some embodiments, the controller 65 (e.g., the I/O devices 92) may be configured to receive voice control commands and/or the display unit 60 may have touchscreen capabilities as an alternative to using the keyboard 62, where the surgeon 15 may use a pointer device, (e.g., an input device 92), for example, to activate the graphical user interface elements on the GUI 61 that are programmed to allow the surgeon 15 to adjust surgical parameters via the display unit 60 during the surgical procedure, as will be shown hereinbelow.

In at least some embodiments not shown in FIG. 1, the controller 65 may be configured to control a surgical robotic assembly that may be used to perform the joint arthroplasty robotically.

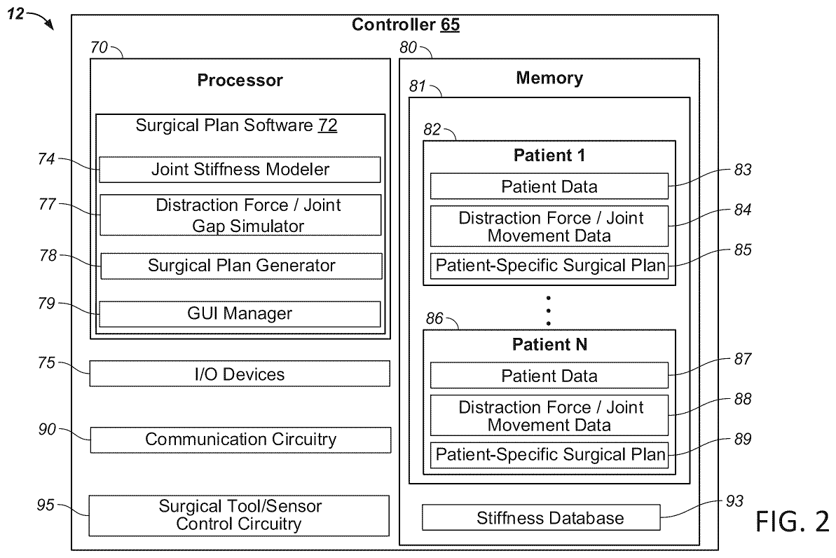

FIG. 2 is a block diagram of the controller 65 of an improved computer-based platform for digitally adjusting joint laxity in a personalized surgical plan of a joint arthroplasty procedure in accordance with one or more embodiments of the present disclosure. The controller 65 of the surgery assistance device 12 represented in FIG. 1 may include a processor 70, a memory 80, input and output (I/O) devices 75 such as the display 60 and the keyboard 62, a communication circuitry 90, and a surgical tool and sensor control circuitry 95. The communication circuitry 90 may enable the controller 65 to communicate with other computing devices over any suitable wired and/or wireless communication network. The communication circuitry 90 may be enabled by the controller 65 to communicate with the at least one surgical tool 56A and/or with the at least one surgical probe 56B, and/or the at least one imaging camera 50 and/or with the at least one first tracker 40A and/or the at least one second tracker 40B.

In at least some embodiments, the surgical tool and sensor control circuitry 95 may be configured to process sensor signals from the at least one surgical tool 56A and the at least one surgical probe 56B, and/or the at least one imaging camera 50 and/or with the at least one first tracker 40A and/or the at least one second tracker 40B, and/or for any other suitable surgical devices and/or sensors needed to perform the total joint arthroscopy procedure. In other embodiments, the surgical tool and sensor control circuitry 95 may be configured to receive commands from the processor 70. The commands may be used to control the at least one surgical tool 56A and the at least one surgical probe 56B during surgery, and/or to control a robotic surgical apparatus for performing the surgical total joint arthroscopy procedure in the operating room 10.

In at least some embodiments, the processor 70 may be configured to execute a surgical plan software 72 that may include at least one software module implemented as algorithms, trained machine learning modules, or both. The at least one software module may include a joint stiffness modeler 74, a joint gap and distraction force simulator 77, a surgical plan generator 78, and/or a GUI manager 79.

In at least some embodiments, the surgical plan generator 78 may be used for generating and/or updating the patient-specific surgical plan in real time so as to assist the surgeon 15 before and/or during the joint arthroplasty procedure. The surgical plan generator 78 may use as inputs any of: joint laxity (joint gap) data under at least one force regime (measured and/or simulated), an implant profile, a surgeon-specific surgery profile, and a patient-specific post-surgery desired functional profile.

In at least some embodiments, the memory 80 may be configured to store a stiffness database 93 and a patient data database 81 storing the data from N patients, where N is an integer. The patient data database 81 may include a patient record 82 of patient 1 that includes for patient 1, patient data 83, distraction force and/or joint movement data 84, and a patient-specific surgical plan 85. The patient data database 81 may include a patient record 86 of the Nth patient N that includes for patient N, patient data 87, distraction force and/or joint movement data 88, and a patient-specific surgical plan 89.

In at least some embodiments, the surgical plan generator 78 executed by the processor 70 The may output a patient-specific surgical plan stored in the patient data database 81. A GUI manager software module 79 may be configured to transmit instructions to the display 60 to display, for example, the patient-specific surgical plan 85 on the GUI 61 for the surgeon 15 to view before and/or during the arthroplasty surgical procedure. All or any of the above software routines may be stored in the memory 80.

In at least some embodiments, bone registration data may be obtained using at least one intra-operative imaging modality such as X-ray imaging, computed tomography (CT) imaging, magnetic resonance imaging (MRI), or any combination thereof.

In at least some embodiments, bone registration data may include geometric points defined along a surface of the bone member which may use medical image data to delineate the bone edge boundaries and other bone features. The bone registration data may be used to model a bone member representation in which the geometric points along the bone edge boundaries and/or other bone features may be defined within a coordinate system. In other embodiments, each bone member representation may be defined in its own unique coordinate system. In yet other embodiments, the first bone member representation may be defined in a single coordinate system.

In at least some embodiments, using the bone registration data, the controller 65 may use a First/Second Bone member representation modeler to generate a first bone member representation of the first bone member and a second bone member representation of the second bone member using the bone registration data.

In at least some embodiments, the surgical plan software 72, as illustrated in FIG. 2, may be stored in the memory 80 and executed by the processor 70 of the controller 65. The surgical plan software 72 may perform functions related to generating the patient-specific intra-operative surgical plan. The software may obtain bone registration data for a first bone member and a second bone member of a joint using input and output (I/O) devices 75 and communication circuitry 90; may acquire, during the surgical procedure, patient-specific movement-related data after the first bone member, the second bone member, or both, have been put through at least one movement where a first predefined distraction force is applied between the first bone member and the second bone member throughout a continuous range of motions, with data received from surgical tools and sensor control circuitry 95.

In at least some embodiments, the surgical plan software 72 may conduct, using a distraction force model, a joint gap simulation (e.g., distraction force/joint gap simulator module 77) based on predefined ligament stiffness data, the patient-specific movement-related data at the first predefined distraction force, and at least one simulated distraction force value; may obtain, from the joint gap simulation, joint gap simulation data; may utilize a surgical plan model (e.g., the surgical plan generator 78) to generate a patient-specific intra-operative surgical plan for the implantation of the implant based at least in part on the joint gap simulation data and the bone registration data for the first bone member and the second bone member; and may control or provide instructions for cutting the first bone member, the second bone member, or both, based at least in part on at least one surgical cut parameter of the patient-specific intra-operative surgical plan to facilitate the implantation of the implant. The generated patient-specific intra-operative surgical plan and related data may be displayed to the surgeon via the display 60 and graphical user interface 61.

In at least some embodiments, the surgical plan software 72 may receive a surgeon-specific surgery profile, a patient-specific post-surgery desired functional profile, implant profiles, bone registration data, patient-specific movement-related data, joint stiffness data from the joint stiffness modeler 74, and/or joint gap data from the distraction force/joint gap simulator 77 as inputs. The surgical plan generator 78, that includes the surgical plan model, may apply these inputs to the surgical plan model to generate the patient-specific intra-operative surgical plan by evaluating at least a plurality of dependencies between surgical parameters, implant characteristics, expected functional performance of the joint, movement-related data, the joint stiffness data, and/or the joint gap data. The model may be designed to achieve the patient-specific post-surgery desired functional profile by determining estimated values for each surgical parameter, such as bone resection depths, alignment angles, and flexion angles, that may be tailored to both the surgeon's preferences and the patient's anatomical and functional requirements.

In at least some embodiments, the surgical plan model may include ranges of acceptable and preferred values for each surgical parameter, which may be defined by the surgeon, the implant manufacturer, or both. The model may process these ranges along with the patient-specific functional objectives, such as targeted joint gaps or alignment, to compute a set of surgical cut parameters that may fall within the defined tolerance bands. The model may also allow for the prioritization or weighting of different functional objectives, so that the surgical plan may reflect the most important clinical outcomes for the patient.

In at least some embodiments, the surgical plan model may be configured to update the patient-specific intra-operative surgical plan in real time as new intra-operative data is acquired. For example, after preliminary bone cuts or soft-tissue assessments, the model may recalculate the optimal surgical parameters based on updated movement-related data or changes in the soft-tissue envelope. The updated plan may be displayed to the surgeon on a graphical user interface, allowing for interactive adjustment of surgical variables and immediate visualization of the predicted impact on joint function.

In at least some embodiments, the surgical plan model may generate the patient-specific intra-operative surgical plan by leveraging machine learning algorithms trained on historical surgical data, patient outcomes, and implant performance. The model may use these data-driven insights to recommend surgical parameters that may optimize both the immediate surgical result and the long-term functional outcome for the patient. The plan may include detailed guidance for bone cuts, implant positioning, and soft-tissue management, and may be presented to the surgeon in a format that highlights key performance indicators such as alignment, balance, and sizing.

In at least some embodiments, the surgical plan model may support the comparison of multiple surgical scenarios by generating alternative patient-specific intra-operative surgical plans based on different input parameters or implant options. The surgeon may review these alternatives on the graphical user interface, compare predicted functional outcomes, and select the most appropriate plan for execution during the procedure.

In at least some embodiments, the surgical plan software 72 may receive, by the controller 65, a patient-specific profile that may include a plurality of patient-specific values for a plurality of patient-specific parameters. These patient-specific parameters may include anatomical measurements, demographic information, preoperative imaging data, and movement-related data unique to the individual patient. The software may use these values as key inputs for generating a surgical plan tailored to the patient's unique anatomy and clinical needs.

In at least some embodiments, the surgical plan software 72 may also receive, by the controller 65, a healthcare-specific profile that may include a plurality of healthcare-specific values for a plurality of healthcare-specific parameters. These healthcare-specific parameters may include surgeon preferences, institutional protocols, implant manufacturer guidelines, and other clinical best practices. The software may use these values to ensure that the generated surgical plan aligns with the standards and requirements of the healthcare provider or institution.

In at least some embodiments, the inputting of the plurality of inputs into the surgical plan model may include inputting both the plurality of patient-specific values for the patient-specific parameters and the plurality of healthcare-specific values for the healthcare-specific parameters into the surgical plan model. The surgical plan model may process these combined inputs to generate the patient-specific intra-operative surgical plan that is both personalized to the patient and consistent with healthcare provider requirements.

In at least some embodiments, the surgical plan model may be designed to achieve the patient-specific post-surgery desired functional profile based at least in part on a plurality of dependencies between any of: the patient-specific parameters, the healthcare-specific parameters, the surgical parameters, at least one functional parameter representative of the expected functional performance of the joint, the movement-related data, the joint stiffness data, and/or the joint gap data. The model may evaluate these dependencies to determine optimal surgical cut parameters, implant selection, and alignment strategies that may maximize the likelihood of achieving the desired clinical and functional outcomes for the patient.

In at least some embodiments, the at least one surgical cut parameter may include a bone resection depth for the first bone member, the second bone member, or both; may include an alignment angle for the bone cuts; may include a flexion angle at which the cuts are performed; may include the thickness of the prosthetic component to be implanted; and may include parameters related to the balancing of soft tissue such as adjustments to achieve targeted joint gaps. The surgical plan model may determine these parameters based on the patient-specific movement-related data, the bone registration data, and the desired functional outcome as well as the joint stiffness data, and/or the joint gap data, so that the resulting patient-specific intra-operative surgical plan may be tailored to the anatomical and clinical needs of the patient.

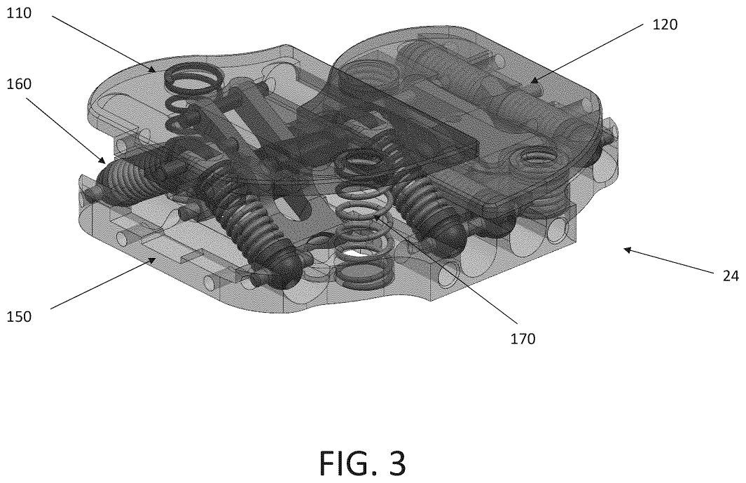

FIG. 3 shows an exemplary embodiment of a distractor 24 in accordance with one or more embodiments of the present disclosure. The distractor 24 may also be referred to herein as a ligament balancing device, a tensor, or a distractor device. The distractor 24 may apply a distraction force between the two bones of the joint, in this exemplary embodiment, the knee joint. In the distractor 24, the spring families 160, 170 cooperate to maintain a substantially constant axial distraction force between the first plate 110 or 120 for interfacing with the femoral medial and lateral condyles, and the second plate 150 for interfacing with the tibia regardless of the distance/height between the first plate 110 or 120 and the second plate 150. The distraction force may be applied by the axial springs 170 along the range of motion of the first plate 110 or 120 to the bone members of the joint for enabling the measurement of movement-related data of the joint.

The exemplary embodiment of the intra-operative distractor device (e.g., the distractor 24) as shown in FIG. 3 is merely for visual and conceptual clarity and not by way of limitation of the embodiments disclosed herein. Any suitable tensor and/or distractor device may be used to apply the distraction force to the joint either intra-operative or pre-surgery so as to measure the movement-related data after the first bone member of the joint, the second bone member of the joint, or both, have been put through a plurality of spatial positions when the distraction force is applied between the first bone member and the second bone member to facilitate obtaining patient-specific movement-related data.

In at least some embodiments, the plurality of spatial positions to obtain the patient-specific movement-related data may be a plurality of spatial positions through a continuous range of motions.

In at least some embodiments, the exemplary embodiment of the distractor 24 as shown in FIG. 3 may supply a predefined distraction force by the first plates 110 and 120 to each of the femoral medial and lateral condyles in the femur 30.

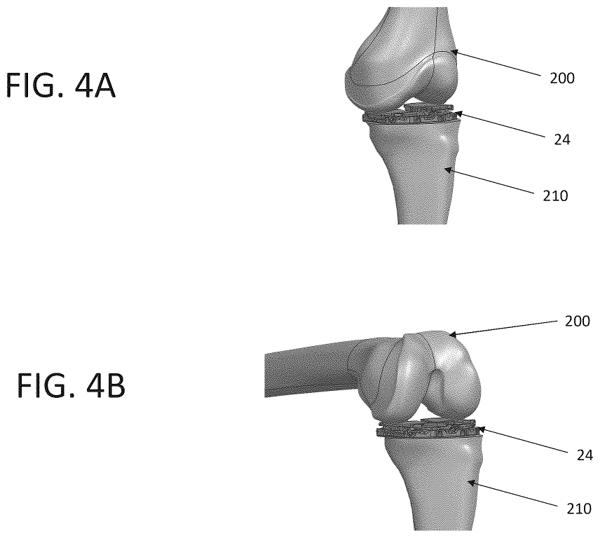

FIGS. 4A and 4B illustrate the distractor 24 inserted between a femur 200 and a tibia 210 in accordance with one or more embodiments of the present disclosure. FIG. 4A shows the leg 25 in extension and FIG. 4B shows the leg 25 in flexion.

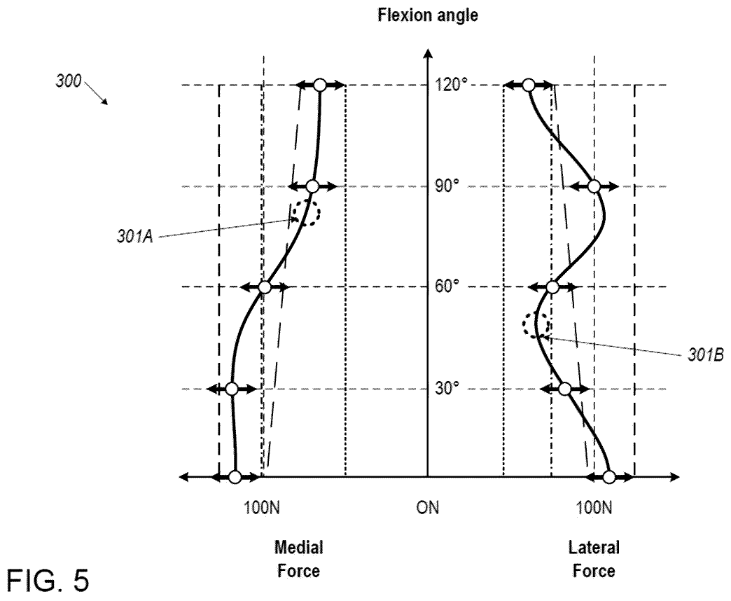

FIG. 5 is a graph 300 of different force environments in accordance with one or more embodiments of the present disclosure, where the X-axis represents the amplitude of the simulated second distraction force for the knee medial compartment on the left and the knee lateral compartment on the right, and the Y-axis represents the flexion angle. The examples of different force regimes or environments may include a low distraction force for both compartments (fine dashed curves), a high distraction force for both compartments (medium coarse dashed curves), a flexion specific distraction force where the force decreases from extension to flexion (coarse dashed curves), a compartment specific distraction force where the force on one compartment (e.g., medial compartment) is higher than the force on the other compartment (e.g., lateral compartment) (dashed-dot curves), a free distraction force shape curve: linear, stepped, spline (solid black curves), or any combination of the force regimes mentioned above where the simulated force may be applied between the first and second bone members.

In at least some embodiments, the graph 300 may be displayed on the GUI 61 and the graph 300 may include slidable cursor/markers 301A and 301B that may be used by the surgeon 15 to set directly on the GUI 61, the value of the second distraction force on the GUI 61. Optionally and/or alternatively, the surgeon 15 may input the second distraction force using any suitable inout device (e.g., keyboard, tablet, etc.).

In at least some embodiments, as illustrated in FIG. 5, the user interface (e.g., the GUI 61) may include features that allow the surgeon to adjust the second distraction force in both qualitative and quantitative ways. For a mainly qualitative approach, the second distraction force may be set lower than the first distraction force for the lateral compartment, but equal for the medial compartment. The user interface may include “−” and “+” cursors next to the first distraction force curve (not shown in FIG. 5), so the surgeon 15 may decrease or increase the second distraction force. These cursors may apply to the entire curve through the full continuous range of motions or may be limited to a defined section of the curve, such as from 30 degrees to 60 degrees of flexion.

In at least some embodiments, a quantitative approach may be used, where the second distraction force may be set lower than the first distraction force for the lateral compartment by a specific value, such as 10 N, but remains equal for the medial compartment. This adjustment may be expressed in absolute terms (for example, the second distraction force is 60 N) or in relative terms (for example, the second distraction force is lower than the first distraction force by 30 N). The user interface (e.g., the GUI 61) may include “−X N” and “+X N” cursors next to the first distraction force curve, where X represents a number ranging from 1 to 100, so the surgeon may decrease or increase the second distraction force by increments of X Newtons or another unit of force. These cursors may be applied to the full curve or to a defined section of the curve. In a hybrid approach, as shown in FIG. 5, the surgeon may slide cursors or markers set at defined locations along the full continuous range of motions to set the second distraction force.

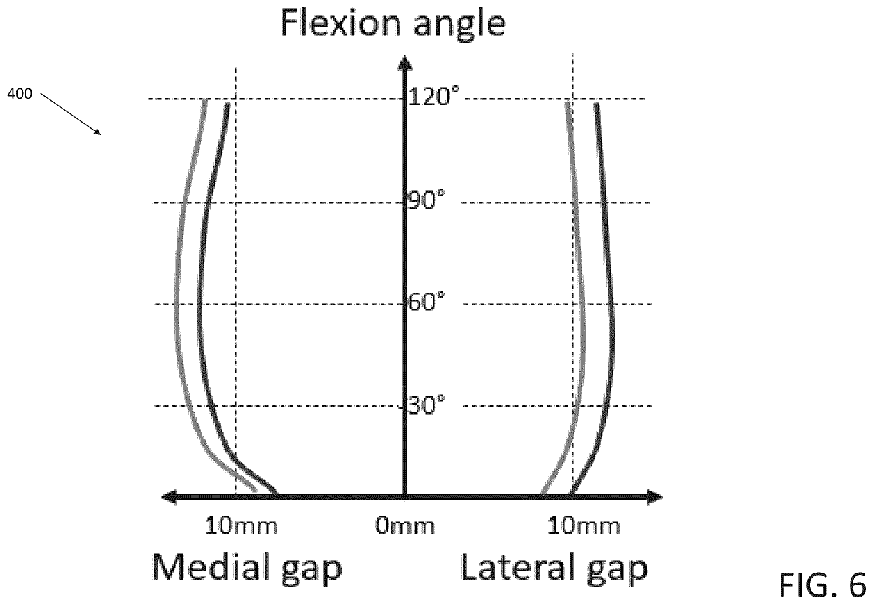

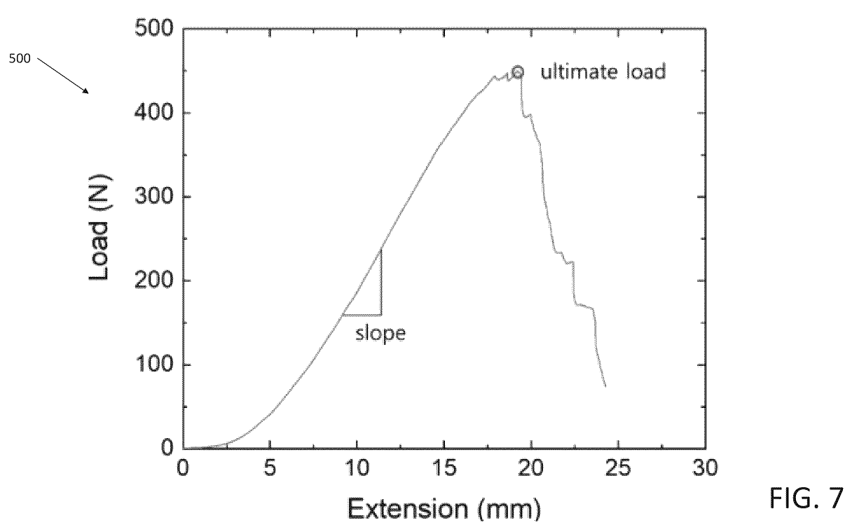

FIG. 6 is a graph 400 showing a first set of joint gaps (black curves) based on physical acquisitions using first distraction force and a personalized second set of joint gaps (grey curves) based on the digital application of a second distraction force in accordance with one or more embodiments of the present disclosure. Specifically, FIG. 6 shows a first set of joint gaps physically acquired under a first known distraction force (black curves) (e.g., a first predefined distraction force) and a second set of joint gaps digitally obtained under a modified second set of personalized distraction force (grey curves) (e.g., with at least one simulated distraction force value).

In at least some embodiments, the processor 70 using the distraction force and joint gap simulator 77 module may use a distraction force model that may be configured to perform the following steps for generating a theoretical set of laxity curves for different distraction force regimes based on measured laxity curves using at least one distractor (e.g., the distractor 24) configured to apply at least one distraction force. The processor 70 may receive a first set of the joint gaps physically acquired by applying a first known distraction force to the joint (e.g., a first predefined distraction force), such as for example, 90 N per compartment. (See FIG. 6—black curves). The processor 70 may receive a stiffness factor of the medial-collateral ligament (MCL) and lateral-collateral ligament (LCL) (e.g., 70±20 N/mm and 50±20 N/mm, respectively) associated with the soft tissue surrounding the joint. These may be referred to herein as ligament stiffness data or ligament stiffness metrics. Assuming a second set of desired medial and lateral distraction forces such as for example, 120 N and 60 N, respectively, digitally obtaining from the distraction force and joint gap simulator 77 module, a simulated second acquisition of the joint gaps under the desired distraction force (See FIG. 6—grey curves). Specifically, on the medial side, the higher second distraction force (e.g., 120 N) than the first distraction force (e.g., 90 N) explains the higher second joint gaps (grey curve for the medial compartment) than the first joint gaps (black curve for the medial compartment). Conversely, on the lateral side, the lower second distraction force (e.g., 60 N) than the first distraction force (e.g., 90 N) explains the lower second joint gaps (grey curve for the lateral compartment) than the first joint gaps (black curve for the lateral compartment).

In at least some embodiments, a plurality of different joint gap data may be theoretically determined for the joint of a patient for different force regimes. This joint gap data acquisitions for the different force regimes and other patient data in terms of alignment and size of the joint may be inputted into the surgical plan generator 78 executing a surgical plan model to output the patient-specific surgical plan 85 (e.g., at least one patient-specific intra-operative surgical plan) for the bone cut parameters that is personalized for a particular patient.

FIG. 7 is a graph 500 that defines a stiffness as a slope of the elongation curve in accordance with one or more embodiments of the present disclosure. The stiffness may be a ratio between the force differential and the displacement differential associated with the soft tissue surrounding the joint.

In at least some embodiments, the processor 70 may use the joint stiffness modeler 74 to determine the stiffness of the joint. The stiffness of the joint may be represented by parameters such as a ligament stiffness value or metric, or predefined ligament stiffness value or metric. The processor 70 may determine the definition of the stiffness associated with the soft tissue surrounding the considered joint, by several methods. The processor 70 may determine the patient's specific stiffness by establishing a patient-specific measured elongation curve (e.g., load (N) versus extension (mm)). The stiffness may be defined as the slope of the elongation curve (i.e., Force differential/Displacement differential) as shown in FIG. 7.

In at least some embodiments, the processor 70 may receive a patient-specific stiffness from a selection from a library of measured stiffnesses associated with patient demography information (e.g., age, gender, stature, . . . ) and pathology (e.g., different advancement of osteoarthritis).

In at least some embodiments, the processor 70 may determine a patient-specific stiffness based on a processing of imaging modalities such as CT or MRI such that the cross section of the soft-tissue may be leveraged as a marker of its stiffness.

In at least some embodiments, the processor 70 may determine a patient-specific stiffness based on musculoskeletal modeling that may assess the joint stiffness with respect to load and muscle tendons specific to the patient. For instance, musculoskeletal modeling software (e.g., AnyBody) may be used to determine optimal joint stiffness based on patient attributes.

In at least some embodiments, the processor 70 may determine a patient-specific stiffness using machine learning algorithms such as clustering. A trained machine learning model for example may determine patient-specific stiffness based on clustering attributes such as patient inputs, pathology, etc. Time series machine learning models may be trained based on past data such as for example, distraction force curve or trajectory that may personalized as surgeon-specific data and/or patient-specific data.

In at least some embodiments, the distraction force per compartment may be between 20-30 N. In at least some embodiments, the distraction force per compartment may be between 30-40 N. In at least some embodiments, the distraction force per compartment may be between 40-50 N. In at least some embodiments, the distraction force per compartment may be between 50-60 N. In at least some embodiments, the distraction force per compartment may be between 60-70 N. In at least some embodiments, the distraction force per compartment may be between 70-80 N. In at least some embodiments, the distraction force per compartment may be between 80-90 N. In at least some embodiments, the distraction force may be between 90-100 N. In at least some embodiments, the distraction force per compartment may be between 100-110 N. In at least some embodiments, the distraction force per compartment may be between 110-120 N. In at least some embodiments, the distraction force per compartment may be between 120-130 N. In at least some embodiments, the distraction force per compartment may be between 130-140 N. In at least some embodiments, the distraction force per compartment may be between 140-150 N. In at least some embodiments, the distraction force per compartment may be between 150-160 N.

In at least some embodiments, the stiffness database 93 may be set-up for expected stiffness. The database or library of expected stiffness for the plurality of patients may be setup based on experimental methods and simulated methods as described hereinbelow. At least some of the exemplary embodiments below describe how patient-specific stiffness models may be determined for the stiffness database 93.

In at least some embodiments, stiffness data for the stiffness database 93 may be obtained by conducting experiments on various cadavers, applying various force regimes using some types of distractor device, and acquiring joint gaps versus force data for each specimen.

In at least some embodiments, the processor 70 may execute the following stiffness algorithm to extract expected stiffness from cadaver testing with the following steps:
I. Data collection:
   a. Apply quasi-constant force across the full flexion arc (for ex: 0° to 120° of flexion) to the cadaver and measure joint gaps at this force setting.
   b. Repeat the process multiple times to gather sufficient joint gaps for the specific force setting.
   c. Repeat steps a and b for multiple force setting with in the experimental force regime. Record joint gaps (medial and lateral) for each applied force setting. This will provide joint gaps for various combinations of forces and flexion angles.
II. Regression analysis:
   a. For each flexion angle, perform linear regression analysis between the measured joint gaps and applied forces.
   b. Determine stiffness as the slope of the regression line, which may represent the load versus gap relationship.
III. Stiffness profiling:
   a. Repeating linear regression fits for all flexion angles throughout the full flexion arc to establish stiffness profiles for both medial and lateral compartments.
IV. Expected stiffness: Determine the expected stiffness by various options that may include:
   a. Selecting the slope associated with the highest R-squared value from the regression across the full flexion arc. For instance, if linear fits may be performed for each flexion angle from 0° to 120°, there may be 121 linear fit slopes and 121 R-squared values.
   b. Averaging the slopes associated with top 10 R-squared values (average of 10 stiffness values corresponding to top 10 highest r-squared values)
   c. Averaging the stiffness across the full range of flexion angles, for example, average of 121 stiffness values corresponding to linear fits for 0° to 120° of flexion.
   d. Surgeon preference: selecting linear fit slope values at extension or flexion or mid-flexion

In at least some embodiments, the system shown in FIG. 2 may include a controller 65 that may be configured to track a first anatomical entity of a first member of a joint and a second anatomical entity of a second member of the joint. The controller 65 may use tracking data obtained from a plurality of movements between at least one first tracker and at least one second tracker, where the tracking data may be acquired during pre-operative, intra-operative, and/or post-operative time periods. This tracking data may be used to generate kinematic data of the joint in a coordinate system during each movement of the joint.

Note that the “anatomical entity” may refer to as an anatomical structure or axis (such as a mechanical axis and/or anatomical plane) that may be tracked or referenced by the system to establish spatial relationships, perform measurements, and/or guide surgical planning and assessment throughout the patient's journey of care.

In at least some embodiments, the system may include the capability to apply a controlled distraction force to the joint using a distractor or similar device, and may measure the resultant opening or displacement of the joint. The controller 65 may process the movement-related data to determine the relationship between the applied force and the measured joint gap, which may allow for the calculation of a stiffness value for the joint. This process may be repeated at different stages along the patient's journey of care to provide a longitudinal assessment of joint stiffness.

In at least some embodiments, the memory 80 of the system may be configured to store a patient data database 81, where the database may include patient-specific data, bone registration and/or joint movement data, and/or surgical plans. The system may store stiffness values and/or related kinematic data acquired at various time points, enabling the controller 65 to track changes in joint stiffness and to identify trends and/or deviations that may be clinically significant for each patient.

In at least some embodiments, the system may include a graphical user interface 61 displayed on the display 60, where clinicians may view the acquired movement-related data, joint gap measurements, and/or calculated stiffness values. The graphical user interface may allow for visualization of the patient's joint function and may support the adjustment of surgical parameters or rehabilitation protocols based on objective, patient-specific data collected throughout the journey of care.

In at least some embodiments, the system may include communication circuitry 90 that may enable the controller 65 to transmit stiffness assessment results and related data to other healthcare providers and/or to cloud-based platforms for further analysis and long-term record keeping. This may support coordinated care, continuous quality improvement, and/or the development of best practices based on aggregated patient data collected across the pre-operative, intra-operative, and/or post-operative stages.

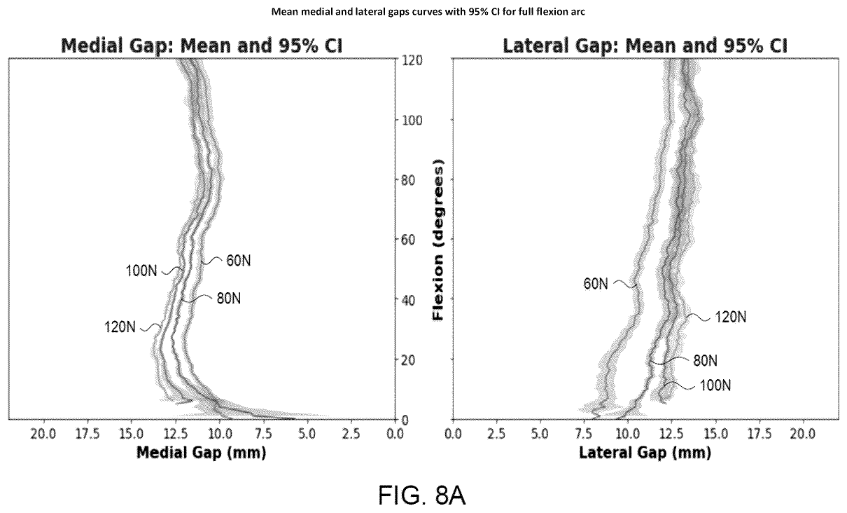

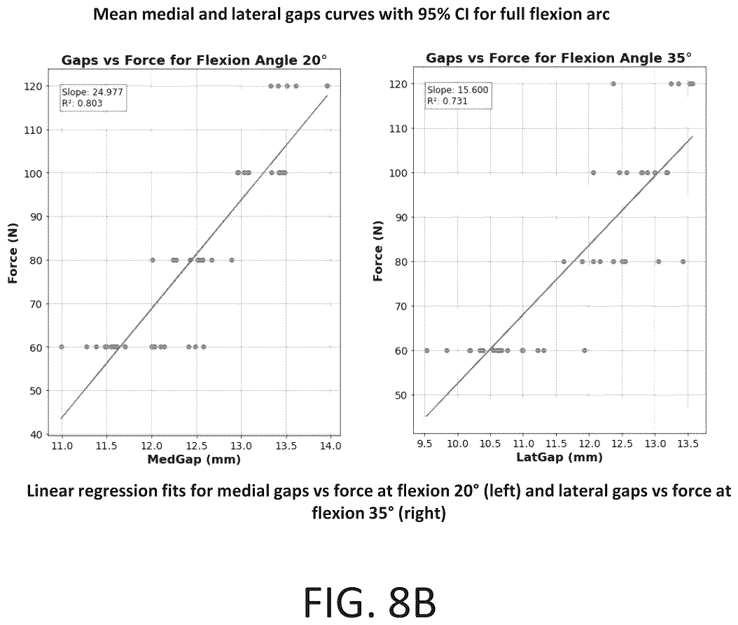

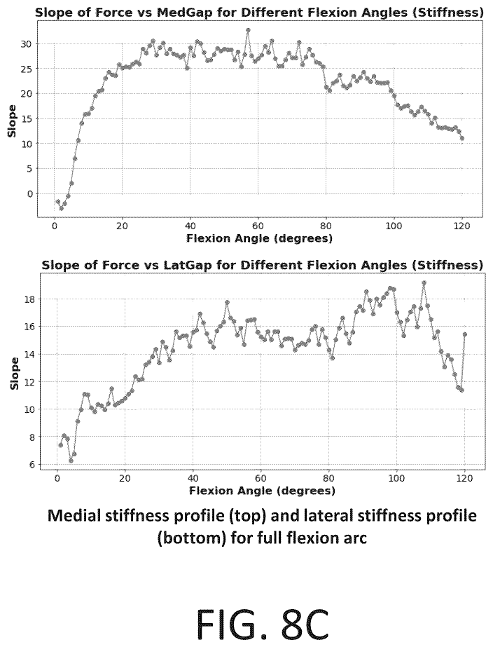

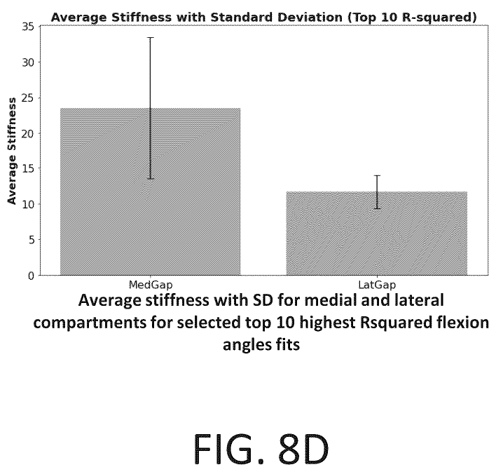

FIGS. 8A-8D are graphs illustrating knee joint medial and lateral stiffness factors calculated from cadaver-based test data in accordance with one or more embodiments of the present disclosure. Medial and lateral joint gaps were acquired multiple times for each quasi-constant force setting across the full flexion arc (0° to 120°), with experiments repeated for forces 60 N, 80 N, 100 N, and 120 N.

FIG. 8A illustrates the mean trajectories of medial and lateral joint gaps with 95% confidence intervals (Cis) for each flexion angle from 0° to 120° and forces 60 N, 80 N, 100 N, and 120 N. Linear regression may be performed using the gaps as inputs and loads as outputs, determining the slope (i.e., stiffness) separately for the medial and lateral gaps.

FIG. 8B are graphs of a linear regression fit for medial gaps (mm) versus loads (60 N to 120 N) at 20° flexion angle and lateral gaps versus loads (60 N to 120 N) at 35° flexion angle. In this way, linear fits may be performed at each flexion angle to determine medial and lateral stiffness profile for full range of flexion.

FIG. 8C shows medial and lateral stiffness profiles across the full range of flexion.

FIG. 8D provides the average medial and lateral stiffness values with standard deviation for cadaver testing. The expected stiffness may be calculated by the stiffness algorithm above (see Step IV). As an example, average stiffness for both medial and lateral compartments may be calculated by selecting top 10 R-squared values fits slopes.

In at least some embodiments, absolute medial and lateral gaps may be taken as inputs, defining the stiffness factor as the slope of load versus absolute medial gap or lateral gap. However, there may be other possibilities for defining stiffness based on surgeon preference. For instance, absolute medial and lateral gaps may be replaced by relative gaps. The relative gaps may be calculated by referencing both the absolute medial and lateral gaps to the medial gap at 0° or 5° of flexion.

In at least some embodiments, stiffness factors may be computed for extension and flexion gaps. The extension gap may be the average of medial and lateral gaps at 0° flexion, while the flexion gap may be the average at 90° flexion. Linear regression of extension gaps versus loads yields extension stiffness, while loads versus flexion gaps yield flexion stiffness. Mid-flexion stiffness may be calculated using 45° flexion gaps versus loads. These experiments can be repeated on numerous cadaver specimens, considering various attributes such as age, gender, and pathology, to build a comprehensive database of specimen-specific stiffness factors. Surgeons can reference this database intraoperatively to define simulated gaps.

In at least some embodiments, the stiffness database 93 may be based on experimental data obtained from conducting experiments on patients in a motion capture lab equipped with infrared cameras and an instrumented treadmill. This setup may capture real-time joint kinematics and ground contact forces during various activities.

In at least some embodiments, systems may provide joint torques by analyzing joint kinematics and ground contact forces, and applying inverse dynamics to a multi-body model to estimate joint torques]. A human knee impedance model may then be used to calculate knee stiffness by analyzing the joint kinematics and kinetics data.

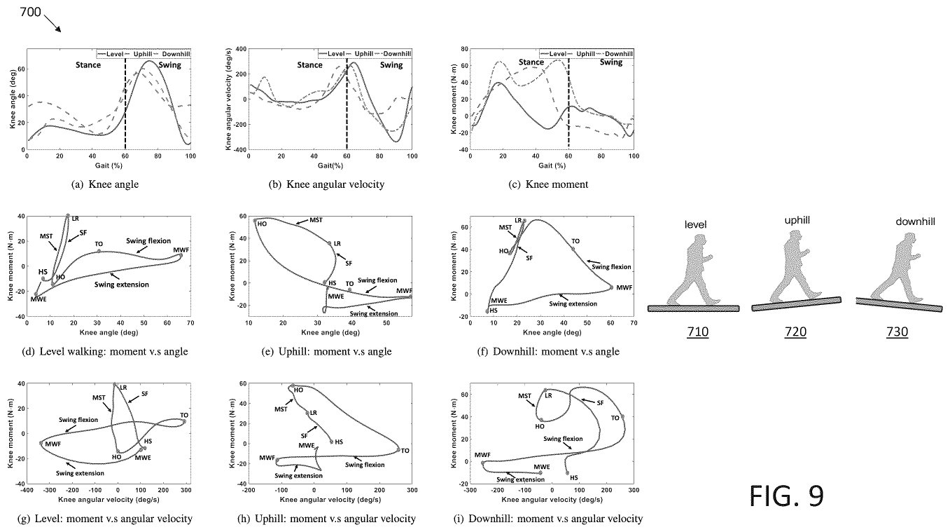

FIG. 9 are plots 700 showing knee torque versus knee angle and knee torque versus knee angular velocity for a participant under different walking conditions in accordance with one or more embodiments of the present disclosure. The data may be generated by experiments on a treadmill to simulate the different walking conditions by adjusting the speed and slope of the treadmill to cover slow, moderate, and fast walking, as well as level 710, uphill 720, and downhill 730 walking as shown in FIG. 9. Linear regression models may be fitted to the linear portions of the plots 700 to calculate stiffness during the stance and swing phases under varying loading conditions.

In at least some embodiments, in addition to walking, participants may perform various daily activities, such as squats and sit-to-stand exercises, using a force mat and infrared cameras. These experiments facilitate an understanding of knee joint dynamics for determining knee joint stiffness under different loading conditions. These experiments may be repeated on numerous participants considering various combination of participant specific attributes such as age, gender, pathology, and other factors. A comprehensive database of participant-specific models and their associated stiffness may then be created. Surgeons may reference this database to define simulated gaps intraoperatively.

In at least some embodiments, another method for determining stiffness for the stiffness database 93 may be include generating data in experiments using a robot and/or a jig. These experiments on patients or healthy participants may be conducted using a knee dynamometer and/or a rotometer, for example, synchronized with fluoroscopy images. This may provide for the collection of data on knee joint torque, knee angle, and/or knee laxity.

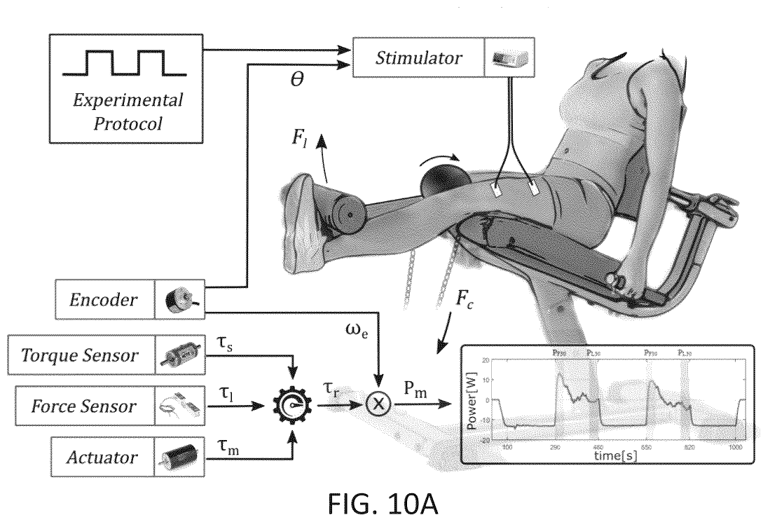

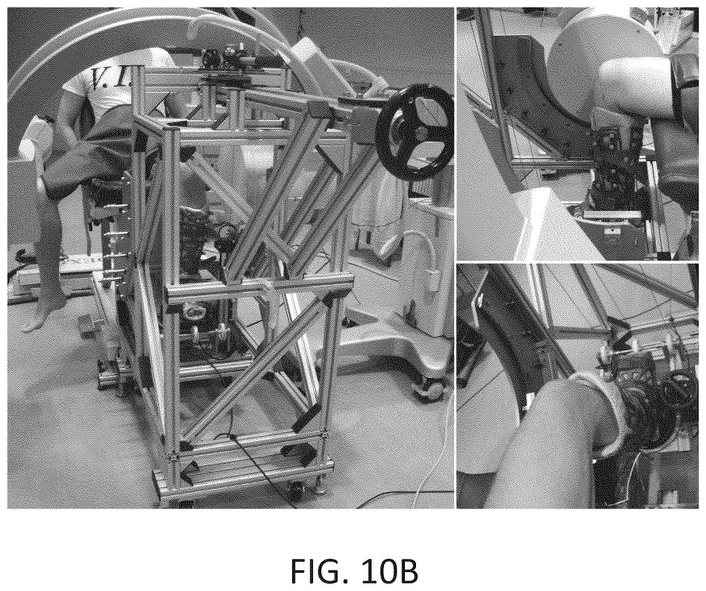

FIGS. 10A and 10B illustrate exemplary embodiments of a knee dynamometer and/or rotometer apparatus in accordance with one or more embodiments of the present disclosure. FIG. 10A shows a system block diagram knee dynamometer and/or rotometer apparatus. FIG. 10B are different views of the knee dynamometer and/or rotometer apparatus. The knee dynamometer may be used to simulate cycling motion with different speed setting along with different torque setting. This may enable the patient to repeat same motion with varying torques, aiding in the determination of knee stiffness.

In at least some embodiments, combining a knee robot such as knee dynamometer and knee rotometer along with fluoroscopy and/or a motion capture system may facilitate the collection of data on a plurality of patients. Utilizing the collect data will help in developing patient-specific models and associated knee stiffness values that may be stored in the stiffness database 93 so as to aid surgeons intraoperatively.

In at least some embodiments, the applied stiffness may be established as a function of the simulated force and/or of the considered flexion angle.

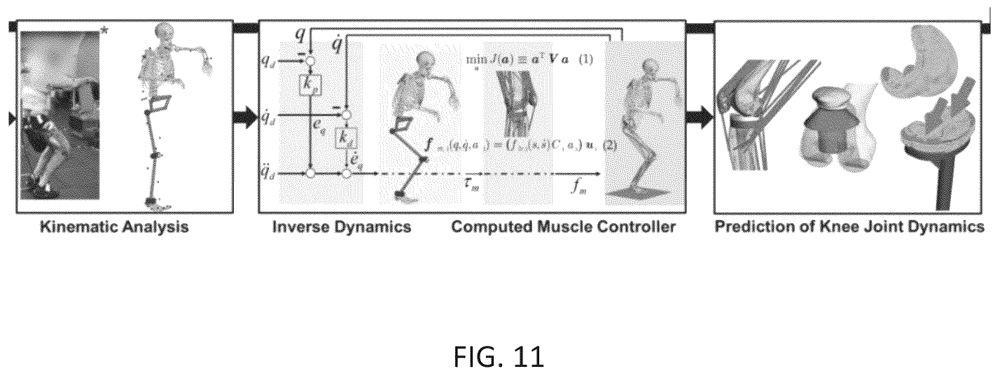

FIG. 11 illustrates the processes for musculoskeletal multibody simulations of a lower extremity with a total knee replacement using a motion capture system in accordance with one or more embodiments of the present disclosure.

In at least some embodiments, the data captured from the motion capture system such as joint kinematic data, ground data captured from motion capture systems, including joint kinematics, ground reaction forces, and/or muscle activation patterns, may serve as a foundation for developing subject-specific musculoskeletal (MS) models. These models, when coupled with finite element (FE) modeling, may provide a robust framework for estimating load distributions, stress, and/or tissue responses within the knee joint, which may be widely used for running gait and/or motion simulations.

In at least some embodiments, MS models may play a role in computational biomechanics for enabling detailed analysis of knee motion under various joint loading conditions. By subjecting the model to controlled forces, such as axial compression, varus-valgus moments, and/or anterior-posterior shear loads, the MS models may be used to predict joint kinematics and to determine a corresponding mechanical response. This process may facilitate the calculation of joint stiffness, which reflects the resistance of the knee to movement under applied forces. Stiffness may typically be computed by assessing the relationship between the change in joint moment and the resulting angular displacement.

In at least some embodiments, by varying input parameters such as ligament tension, muscle forces, and/or external loads, these simulations may be repeated across a wide range of conditions. This iterative process may generate a large volume of stiffness data for creating a comprehensive patient-specific database. Thus, the stiffness database 93 may store stiffness values across different knee joint configurations, loading conditions, and/or patient demographics. The stiffness database 93 that includes simulated stiffness values may then be leveraged by surgeons to predict expected joint behavior and/or stiffness patterns during surgery.

In at least some embodiments, after establishing a comprehensive database of expected stiffness (e.g., the stiffness database 93) through experimental and simulated methods, the data in the stiffness database 93 may be used by the joint stiffness modeler 74 to generate predictive models that can output expected stiffness for a specific patient that surgeon may use intraoperatively to simulate a second definition of the joint gaps to be used for the set-up of the surgical planning using the surgical plan generator 78. Machine learning (ML) methods such as clustering, classification, and/or regression may be utilized to develop patient-specific stiffness models.

In at least some embodiments with regard to inputs for the ML models in the joint stiffness modeler 74, input data may be used from various data sources for training datasets to predict stiffness outputs such as medial stiffness, lateral stiffness, extension stiffness, mid-flexion stiffness, and/or flexion stiffness, for example.

In at least some embodiments, the various data sources may include, but are not limited to:
Patient demographic factors: age, gender, BMI, height, weight, physical activity level, etc.
Anatomic data: joint morphology (femur size, tibia size, knee alignment), and ligament laxity, etc.
Surgical data: type of procedure, preoperative diagnosis, ligament tension, preoperative gap measurements (first definition of joint gaps acquired by physically applying a first known distraction force to the joint).
Gait or kinematics data: kinematics, kinetics, ground contact forces, muscle activation data collected through motion capture systems or wearables preoperatively.
Other inputs of interest

In at least some embodiments, the joint stiffness modeler 74 may use clustering models utilizing clustering techniques such as K-means or hierarchical clustering, which may be applied to group patients with similar stiffness profiles. The stiffness database 93 may be segmented into clusters based on input variables such as anatomical features, age, gender, and/or pathology, for example. Patient comparable characteristics may fall into the same clusters, and stiffness patterns may be computed for each group. This approach may be helpful for identifying general trends and/or outliers which may assist surgeons in determining whether a new patient falls into a high-stiffness or low-stiffness group.

In at least some embodiments, at least one classification model may be used to categorize patients based on stiffness thresholds. For example, based on medial and lateral stiffness data from the stiffness database 93, the at least one classification model may classify patients into predefined categories such as “normal stiffness”, “increased medial stiffness”, or “increased lateral stiffness”. Classification algorithms such as decision trees, random forests, and/or support vector machines (SVM) may be trained to predict these categories using patient-specific inputs. This may provide a surgeon with a real-time categorical prediction of expected knee behavior during surgery with an expected stiffness.

In at least some embodiments, at least one regression model may be used for generating continuous predictions of stiffness outputs (medial, lateral, extension, mid-flexion, and flexion, etc.). By applying linear regression models, non-linear regression models, and/or more advanced techniques like neural networks, the at least one regression model may be trained to output specific stiffness values rather than categories. For instance, given the input variables, the at least one regression model may predict stiffness for allowing surgeons to anticipate joint behavior for full flexion arc.

In at least some embodiments, at least one time series machine learning model may be trained based on past data such as for example, distraction force curve and/or trajectory data that may personalized as surgeon-specific data and/or patient-specific data. Time series models such as Autoregressive Integrated Moving average (ARIMA) and long short time memory (LSTM) networks may be applied to forecast stiffness and force trajectory based on past data.

In at least some embodiments, once the prediction models are trained, they may be integrated into intraoperative decision-making tools in the operating room. Surgeons may input, via the GUI 61 and I/O devices 75, patient-specific attributes to the platform, which may output stiffness values in real-time. These stiffness values may be leveraged to simulate definition of the joint gaps to be used for the set-up of the surgical planning.

In at least some embodiments, the patient-specific surgical plan interface may display a patient-specific surgical plan generated by the surgical plan generator 78.

In at least some embodiments, the method for performing a joint replacement between two bone members may include using a computer aided orthopedic surgery system to tracking a position of the first bone member relative to the second bone member (or vice versa) with a 3D position tracking system (e.g., the at least one first and second trackers 40A and 40B). The processor 70 of the computer aided orthopedic surgery system may use the surgical plan generator 78 of the surgical plan software 72 for the definition of the cut parameters associated with at least one of two bone members.

In at least some embodiments, using a distractor device such as the distractor 24 to applying a known distraction force (F1) at each known spatial position (e.g., the plurality of spatial positions) between the first bone member and the second bone member, resulting in a measured joint gap (G1) at each of the known spatial positions between the first bone member and the second bone member. The known distraction force (F1) may be referred to herein interchangeably as a measured distraction force, a physical distraction force, or a (first) predefined distraction force.

In at least some embodiments, the processor 70 using the joint stiffness modeler 74 of the computer aided orthopedic surgery system may determine a known stiffness(S) of the considered soft tissue surrounding the joint.

In at least some embodiments, the processor 70 using the distraction force joint gap simulator 77 with at least one virtual distraction force (Fi) to determine an updated joint gap (Gi) at each known spatial position between the first bone member and the second bone member using the equation Gi=G1+(Fi−F1)/S that may be referred to as a joint gap simulation model for performing a joint gap simulation. The at least one virtual distraction force may be referred to as at least one simulated distraction force.

In at least some embodiments, the processor 70 may leverage the updated joint gap (Gi) to feed the surgical planning application (SPi) (e.g., the surgical plan generator 78) to output the cut parameters of at least one of two bone members; where Gi represents a predicate to establish the thickness of the prosthetic component(s) to be implanted into the considered joint of interest.

In at least some embodiments, the processor 70 using the joint stiffness modeler 74 of the computer aided orthopedic surgery system may determine a tolerance (e.g., based on standard deviation) associated with the stiffness S of the considered soft tissue to obtain a range of gap Gi. In at least some embodiments, the processor 70 using the joint stiffness modeler 74 of the computer aided orthopedic surgery system may assign different stiffnesses to different soft-tissue portions of the considered joint, such as for example S_{med} for the stiffness of the medio-collateral ligament of a knee joint, and Sₗₐₜ for the stiffness of the lateral-collateral ligament of a knee joint.

In at least some embodiments, the processor 70 using the distraction force joint gap simulator 77 and the surgical plan generator 78 may defining a plurality of distraction force regimes Fi (e.g., F2, F3, F4, . . . ) to compute a respective plurality of joint gaps Gi (e.g., G2, G3, G4, . . . ) so as to generate a plurality of surgical plans SPi (e.g., SP2, SP3, SP4, . . . ) that may be outputted for example on the GUI 61. This may allow the user (e.g., the surgeon) to perform direct comparisons between the plurality of surgical plans.

In at least some embodiments, the processor 70 may record the final selected distraction force option as part of surgical planning records such as in the patient data database 81.

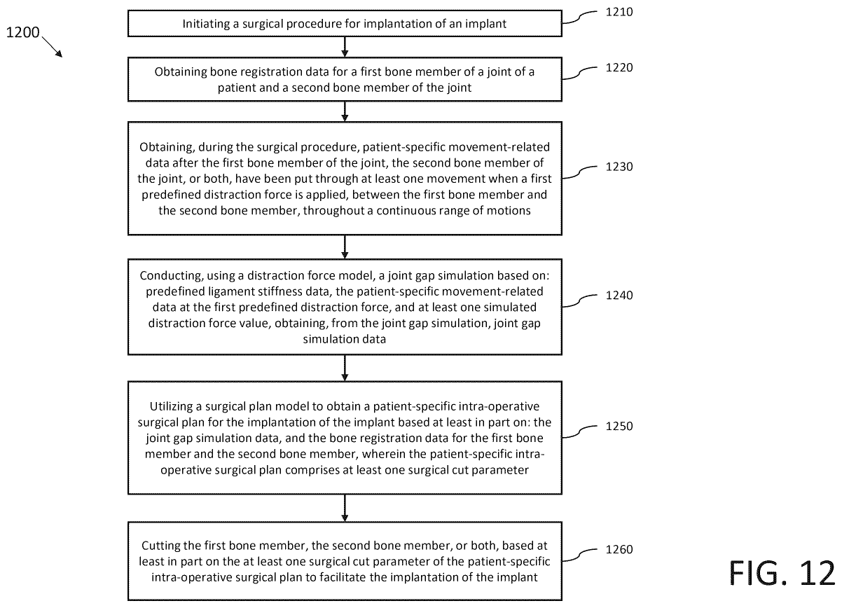

FIG. 12 is a flowchart of an exemplary method 1200 for digitally adjusting joint laxity in a personalized surgical plan of a joint arthroplasty procedure in accordance with one or more embodiments of the present disclosure.

In at least some embodiments, the method 1200 may include initiating 1210 a surgical procedure for implantation of an implant.

In at least some embodiments, the method 1200 may include obtaining 1220 bone registration data for a first bone member of a joint of a patient and a second bone member of the joint.

In at least some embodiments, the method 1200 may include obtaining 1230, during the surgical procedure, patient-specific movement-related data after the first bone member of the joint, the second bone member of the joint, or both, have been put through at least one movement when a first predefined distraction force is applied, between the first bone member and the second bone member, throughout a continuous range of motions.

In at least some embodiments, the method 1200 may include conducting 1240, using a distraction force model, a joint gap simulation based on: predefined ligament stiffness data, the patient-specific movement-related data at the first predefined distraction force, and at least one simulated distraction force value, obtaining, from the joint gap simulation, joint gap simulation data.

In at least some embodiments, the method 1200 may include utilizing 1250 a surgical plan model to obtain a patient-specific intra-operative surgical plan for the implantation of the implant based at least in part on: the joint gap simulation data, and the bone registration data for the first bone member and the second bone member, wherein the patient-specific intra-operative surgical plan comprises at least one surgical cut parameter.

In at least some embodiments, the method 1200 may include cutting 1260 the first bone member, the second bone member, or both, based at least in part on the at least one surgical cut parameter of the patient-specific intra-operative surgical plan to facilitate the implantation of the implant.

In at least some embodiments, a method may include initiating a surgical procedure for implantation of an implant, may obtain bone registration data for a first bone member of a joint of a patient and a second bone member of the joint, may obtain, during the surgical procedure, patient-specific movement-related data after the first bone member of the joint, the second bone member of the joint, or both, may have been put through at least one movement where a first predefined distraction force may be applied, between the first bone member and the second bone member, throughout a continuous range of motions, may conduct, using a distraction force model, a joint gap simulation based on predefined ligament stiffness data, the patient-specific movement-related data at the first predefined distraction force, and at least one simulated distraction force value, may obtain, from the joint gap simulation, joint gap simulation data, may utilize a surgical plan model to obtain a patient-specific intra-operative surgical plan for the implantation of the implant based at least in part on the joint gap simulation data and the bone registration data for the first bone member and the second bone member, where the patient-specific intra-operative surgical plan may include at least one surgical cut parameter, and may cut the first bone member, the second bone member, or both, based at least in part on the at least one surgical cut parameter of the patient-specific intra-operative surgical plan to facilitate the implantation of the implant.

In at least some embodiments, the patient-specific movement-related data may include joint gap measurements obtained at a plurality of flexion angles throughout a continuous range of motion.

In at least some embodiments, the first predefined distraction force may be applied using a distractor device configured to maintain a substantially constant axial force between the first bone member and the second bone member.

In at least some embodiments, the distraction force model may utilize a ligament stiffness value determined by performing linear regression analysis of load versus joint gap data.

In at least some embodiments, the predefined ligament stiffness data may be selected from a database of measured stiffness values associated with patient demographic information.

In at least some embodiments, the joint may be a knee gap, and the conducting of the joint gap simulation may include calculating simulated joint gaps for a plurality of distraction force regimes that include at least one compartment-specific distraction force and at least one flexion-specific distraction force.

In at least some embodiments, the surgical plan model may be configured to generate and update the patient-specific intra-operative surgical plan in real time.

In at least some embodiments, the patient-specific intra-operative surgical plan may include implant selection parameters determined based at least in part on the joint gap simulation data and the bone registration data.

In at least some embodiments, the at least one surgical cut parameter may include a bone resection depth for the first bone member, the second bone member, or both.

In at least some embodiments, cutting the first bone member, the second bone member, or both, may be performed using a robotic surgical apparatus.

In at least some embodiments, the patient-specific movement-related data may be obtained after the joint may be subjected to a plurality of spatial positions using a motion capture system.

In at least some embodiments, simulated joint gaps may be generated using the distraction force model by adjusting the patient-specific movement-related data that may include measured joint gaps obtained at the first predefined distraction force according to differences between the first predefined distraction force and at least one simulation distraction force value and based at least in part on the predefined ligament stiffness data.

In at least some embodiments, the predefined ligament stiffness data may be determined by musculoskeletal modeling based on patient-specific imaging data.

In at least some embodiments, the patient-specific intra-operative surgical plan may be displayed to a surgeon via a graphical user interface during the surgical procedure.

In at least some embodiments, a final selected distraction force option may be recorded as part of surgical planning records.

In at least some embodiments, the joint may be a knee joint, and the distraction force model may assign different stiffness values to different soft-tissue portions of the knee joint that include a medio-collateral ligament and a lateral-collateral ligament.

In at least some embodiments, a plurality of patient-specific intra-operative surgical plans may be generated using the surgical plan model based on different simulated distraction force scenarios, and the plurality of patient-specific intra-operative surgical plans may be output on a graphical user interface for direct comparison by a surgeon.

In at least some embodiments, the bone registration data may be obtained using at least one intra-operative imaging modality selected from the group consisting of computed tomography (CT) and magnetic resonance imaging (MRI).

In at least some embodiments, the patient-specific movement-related data may include joint torque and angular velocity measurements acquired during simulated daily activities.

In at least some embodiments, the second distraction force may be determined by extracting data from the processing of previous surgical logs (e.g., from the patient database in FIG. 2 for the plurality of N patients—the patient 1 82 . . . the Nth patient 86). By knowing the first, or initial, joint gaps measured under the first distraction force and the second, or final, joint gaps defined through the surgical planning, and/or by assuming a joint stiffness, the system may calculate the second distraction force using the joint stiffness modeler 74 and/or the distraction force/joint gap simulator 77. This approach may allow for the second distraction force to be inferred based on historical intra-operative data and outcomes.

In at least some embodiments, the second distraction force may be further tailored depending on different parameters associated with the patient, such as demographic information, phenotype, and/or patient expectations, as well as the surgeon's individual preferences. The definition of the second distraction force may follow a learning process loop, where the system may continuously refine and/or personalize the distraction force based on accumulated data from prior procedures and/or evolving clinical insights.

In at least some embodiments, a real-time graphical representation of the simulated joint gaps may be displayed to a surgeon on a graphical user interface during the surgical procedure.

In at least some embodiments, the patient-specific intra-operative surgical plan and simulated joint gap data may be presented to the surgeon using a mixed reality (MR) and/or augmented reality (AR) system. The system may include a head-mounted AR headset that may overlay digital representations of the predicted joint gaps directly onto the surgical field (e.g., physical area of the patient's knee 20 that is exposed and prepared for the surgical procedure by the surgeon 15). The overlay may include color-coded indicators, where in an exemplary embodiment, green may indicate balanced gaps, red may indicate excessive tightness, and blue may indicate excessive laxity. The system may further display on the headset, comparative heatmaps that may show predicted versus measured joint gaps across the range of flexion, visualized as gradient bars and/or three-dimensional plots.

In at least some embodiments, the overlaying information overlaid directly onto the surgical field may refer to the digital data, such as predicted joint gaps, color-coded indicators, and/or heatmaps, that may be visually superimposed onto the real-time view of the patient's anatomy as seen by the surgeon through a head-mounted display or similar device, and may assist the surgeon in surgical planning and execution.

In at least some embodiments, the processor 70 may transmit commands to an augmented reality headset to cause it to display, for example, the patient-specific intra-operative surgical plan the simulated joint gap data, and/or any other suitable data as described herein, as a superimposed overlay in a view of the surgical field via the augmented reality headset.

In at least some embodiments, the MR and/or AR system may allow the surgeon to interact with the visualization in a hands-free manner. The surgeon may use gestures, foot pedals, and/or voice commands to cycle between different distraction force simulations and/or implant positioning options. This interactive visualization may support real-time decision-making and may enhance the surgeon's ability to assess and optimize joint balance during the procedure.

In at least some embodiments, exemplary inventive, specially programmed computing systems/platforms with associated devices (e.g., the surgery assistance device 12) are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In at least some embodiments, the NFC can represent a short-range wireless communications technology in which NFC-enabled devices are “swiped,” “bumped,” “tap” or otherwise moved in close proximity to communicate. In at least some embodiments, the NFC could include a set of short-range wireless technologies, typically requiring a distance of 10 cm or less. In at least some embodiments, the NFC may operate at 13.56 MHz on ISO/IEC 18000-3 air interface and at rates ranging from 106 kbit/s to 424 kbit/s. In at least some embodiments, the NFC can involve an initiator and a target; the initiator actively generates an RF field that can power a passive target. In at least some embodiments, this can enable NFC targets to take very simple form factors such as tags, stickers, key fobs, or cards that do not require batteries. In at least some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In at least some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In at least some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In at least some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In at least some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21).NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In at least some embodiments, exemplary inventive computer-based systems/platforms, exemplary inventive computer-based devices, and/or exemplary inventive computer-based components of the present disclosure (e.g., the surgery assistance device 12) may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In at least some embodiments, exemplary inventive computer-based systems/platforms, exemplary inventive computer-based devices, and/or exemplary inventive computer-based components of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In at least some embodiments, exemplary inventive computer-based systems/platforms, exemplary inventive computer-based devices, and/or exemplary inventive computer-based components of the present disclosure (e.g., the surgery assistance device 12) may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display (e.g., for the surgeon 15) may be a holographic display. In various implementations, the display may be a transparent surface that may receive a visual projection. Such projections may convey various forms of information, images, and/or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Pager, Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform of the present disclosure and/or any associated computing devices” (e.g., for tracking movements of the leg 25), based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In at least some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

As used herein, the term “user” shall have a meaning of at least one user. In at least some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

In at least some embodiments, the exemplary inventive computer-based systems/platforms, the exemplary inventive computer-based devices, and/or the exemplary inventive computer-based components of the present disclosure (e.g., the software modules implemented in the surgery assistance device 12) may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In at least some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In at least some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In at least some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In at least some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated.

In at least some embodiments and, optionally, in combination of any embodiment described above or below, the exemplary aggregation function may be a mathematical function that combines (e.g., sum, product, etc.) input signals to the node. In at least some embodiments and, optionally, in combination of any embodiment described above or below, an output of the exemplary aggregation function may be used as input to the exemplary activation function. In at least some embodiments and, optionally, in combination of any embodiment described above or below, the bias may be a constant value or function that may be used by the aggregation function and/or the activation function to make the node more or less likely to be activated.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. A method, comprising:
initiating a surgical procedure for implantation of an implant;
obtaining bone registration data for a first bone member of a joint of a patient and a second bone member of the joint;
obtaining, during the surgical procedure, patient-specific movement-related data after the first bone member of the joint, the second bone member of the joint, or both, have been put through at least one movement when a first predefined distraction force is applied, between the first bone member and the second bone member, throughout a continuous range of motions;
conducting, using a distraction force model, a joint gap simulation based on:
predefined ligament stiffness data,
the patient-specific movement-related data at the first predefined distraction force, and
at least one simulated distraction force value;
obtaining, from the joint gap simulation, joint gap simulation data;
utilizing a surgical plan model to obtain a patient-specific intra-operative surgical plan for the implantation of the implant based at least in part on:
the joint gap simulation data, and
the bone registration data for the first bone member and the second bone member;
wherein the patient-specific intra-operative surgical plan comprises at least one surgical cut parameter; and
cutting the first bone member, the second bone member, or both, based at least in part on the at least one surgical cut parameter of the patient-specific intra-operative surgical plan to facilitate the implantation of the implant.

2. The method of claim 1, wherein the patient-specific movement-related data comprises joint gap measurements obtained at a plurality of flexion angles throughout a continuous range of motion.

3. The method of claim 1, wherein the first predefined distraction force is applied using a distractor device configured to maintain a substantially constant axial force between the first bone member and the second bone member.

4. The method of claim 1, wherein the distraction force model utilizes a ligament stiffness value determined by performing linear regression analysis of load versus joint gap data.

5. The method of claim 1, wherein the predefined ligament stiffness data is selected from a database of measured stiffness values associated with patient demographic information.

6. The method of claim 1, wherein the joint is a knee gap, and wherein the conducting of the joint gap simulation comprises calculating simulated joint gaps for a plurality of distraction force regimes that include at least one compartment-specific distraction force and at least one flexion-specific distraction force.

7. The method of claim 1, wherein the surgical plan model is configured to generate and update the patient-specific intra-operative surgical plan in real time.

8. The method of claim 1, wherein the patient-specific intra-operative surgical plan comprises implant selection parameters determined based at least in part on the joint gap simulation data and the bone registration data.

9. The method of claim 1, wherein the at least one surgical cut parameter comprises a bone resection depth for the first bone member, the second bone member, or both.

10. The method of claim 1, wherein cutting the first bone member, the second bone member, or both, is performed using a robotic surgical apparatus.

11. The method of claim 1, wherein the patient-specific movement-related data is obtained after the joint is subjected to a plurality of spatial positions using a motion capture system.

12. The method of claim 1, further comprising generating simulated joint gaps using the distraction force model by adjusting the patient-specific movement-related data that comprises measured joint gaps obtained at the first predefined distraction force according to differences between the first predefined distraction force and at least one simulation distraction force value and based at least in part on the predefined ligament stiffness data.

13. The method of claim 1, wherein the predefined ligament stiffness data is determined by musculoskeletal modeling based on patient-specific imaging data.

14. The method of claim 1, wherein the patient-specific intra-operative surgical plan is displayed to a surgeon via a graphical user interface during the surgical procedure.

15. The method of claim 1, further comprising recording a final selected distraction force option as part of surgical planning records.

16. The method of claim 1, wherein the joint is a knee joint, and wherein the distraction force model assigns different stiffness values to different soft-tissue portions of the knee joint that include a medio-collateral ligament and a lateral-collateral ligament.

17. The method of claim 1, further comprising generating a plurality of patient-specific intra-operative surgical plans using the surgical plan model based on different simulated distraction force scenarios; and
outputting the plurality of patient-specific intra-operative surgical plans on a graphical user interface for direct comparison by a surgeon.

18. The method of claim 1, wherein the bone registration data is obtained using at least one intra-operative imaging modality selected from the group consisting of computed tomography (CT) and magnetic resonance imaging (MRI).

19. The method of claim 1, wherein the patient-specific movement-related data includes joint torque and angular velocity measurements acquired during simulated daily activities.

20. The method of claim 1, further comprising displaying, during the surgical procedure, a real-time graphical representation of the simulated joint gaps to a surgeon on a graphical user interface.

21. The method of claim 1, further comprising displaying the patient-specific intra-operative surgical plan and the joint gap simulation as a superimposed overlay in a view of a surgical field via an augmented reality headset.
